(19) United States
(12) Patent Application Publication
(10) Pub. No.: US 2017/0130169 A1
(43) Pub. Date: May 11, 2017

(54) CLEANING AGENT CONTAINING PROTEASES

(71) Applicant: Henkel AG & Co. KGaA, Duesseldorf (DE)

(72) Inventors: Thomas Eiting, Duesseldorf (DE); Nina Mussmann, Willich (DE); Thorsten Bastigkeit, Wuppertal (DE); Konstantin Benda, Mettmann (DE); Timothy O'Connell, Landsberg am Lech (DE); Hendrik Hellmuth, Darmstadt (DE); Silke Menke, Haan (DE)

(73) Assignee: Henkel AG & Co. KGaA, Duesseldorf (DE)

(21) Appl. No.: 15/323,264

(22) PCT Filed: Jun. 18, 2015
(86) PCT No.: PCT/EP2015/063665

§ 371 Date: Dec. 30, 2016

(30) Foreign Application Priority Data
Jun. 30, 2014 (DE) 10 2014 212 639.8

(51) Int. Cl. C11D 3/386; C11D 17/00; C12N 9/48; C11D 11/00

(52) U.S. Cl. CPC C11D 3/38609; C11D 11/0023; C11D 17/0047; C12N 9/48; C12Y 304/00

(57) ABSTRACT

A cleaning agent is proposed that includes a first and a second protease, wherein a) the first protease comprises an amino acid sequence which is at least 80% identical to the amino acid sequence specified in SEQ ID NO. 1 over the entire length and has at least one amino acid substitution at one of the positions 9, 15, 66, 212, and 239 using the numbering according to SEQ ID NO. 1, and b) the second protease comprises an amino acid sequence which is at least 80% identical to the amino acid sequence specified in SEQ ID NO. 2 over the entire length and has at least one amino acid substitution at one of the positions 32, 33, 48-54, 58-62, 94-107, 116, 123-133, 150, 152-156, 158-161, 164, 169, 175-186, 197, 198, and 203-216 using the numbering according to SEQ ID NO. 2.

### CROSS-REFERENCE TO RELATED APPLICATION

This application is a U.S. National-Stage entry under 35 U.S.C. §371 based on International Application No. PCT/EP2015/063665, filed Jun. 18, 2015, which was published under PCT Article 21(2) and which claims priority to German Application No. 10 2014 212 639.8, filed Jun. 30, 2014, which are all hereby incorporated in their entirety by reference.

### TECHNICAL FIELD

The present disclosure relates to a cleaning agent, preferably a dishwashing agent, preferably a machine dishwashing agent, that contains at least two proteases, as well as to the use of such a cleaning agent.

### BACKGROUND

The most important criterion when cleaning textiles, hard surfaces, such as in particular when washing dishes, in particular during machine dish washing, is the cleaning power when it comes to a wide variety of soiling, which is introduced, in particular, in the form of food residue. While the cleaning power of dishwashing agents used today is generally high, a problem that arises, due also to the general trend in machine dishwashing to increasingly use low-temperature programs, is that many of the usual machine dishwashing agents exhibit inadequate cleaning power on tenacious soiling that has been burnt on. Such inadequate cleaning power and the therewith-resulting inadequate cleaning of the dishes result in dissatisfaction on the part of the consumer and in the consumer pretreating the tenacious soiling, which in turn increases the consumption of water and energy. There is therefore a general need for machine dishwashing agents that have favorable cleaning power even on tenacious soling, in particular, soiling that has been burnt on, but without thereby reducing existing favorable cleaning power on other forms of soiling.

### BRIEF SUMMARY

Cleaning agents and methods for cleaning dishes using the cleaning agents are provided herein. In an embodiment, a cleaning agent includes at least one first protease and one second protease. The first protease includes an amino acid sequence which is at least about 80% identical to the amino acid sequence specified in SEQ ID NO. 1 over the entire length and has at least one amino acid substitution at one of the positions 9, 15, 66, 212, and 239 using the numbering according to SEQ ID NO. 1. The second protease includes an amino acid sequence which is at least about 80% identical to the amino acid sequence specified in SEQ ID NO. 2 over the entire length and has at least one amino acid substitution at one of the positions 32, 33, 48-54, 58-62, 94-107, 116, 123-133, 150, 152-156, 158-161, 164, 169, 175-186, 197, 198, and 203-216 using the numbering according to SEQ ID NO. 2.

### DETAILED DESCRIPTION

The following detailed description is merely exemplary in nature and is not intended to limit the cleaning agents and methods for cleaning dishes using the cleaning agents. Furthermore, there is no intention to be bound by any theory presented in the preceding background or the following detailed description.

The present disclosure addresses the problem of providing a cleaning agent, preferably a dishwashing agent, preferably a machine dishwashing agent, that has an increased cleaning power against such soiling, without a reduction in the cleaning power on other forms of soiling.

Surprisingly, it has now been established that the use of a combination of different proteases considerably improves the cleaning power of corresponding cleaning agents, preferably of a dishwashing agent, preferably a machine dishwashing agent, on protease-sensitive stains, in particular burnt food soiling, in particular burnt sugar-containing food soiling.

In one aspect, therefore, the present disclosure relates to a cleaning agent for hard surfaces, in particular, a cleaning agent, preferably a machine dishwashing agent, that comprises a first protease and a second protease, wherein:
a) the first protease comprises an amino acid sequence which is at least about 80% identical to the amino acid sequence specified in SEQ ID NO. 1 over the entire length and has at least one amino acid substitution at one of the positions 9, 15, 66, 212, and 239 using the numbering according to SEQ ID NO. 1, and
b) the second protease comprises an amino acid sequence which is at least 80% identical to the amino acid sequence specified in SEQ ID NO. 2 over the entire length and has at least one amino acid substitution at one of the positions 32, 33, 48-54, 58-62, 94-107, 116, 123-133, 150, 152-156, 158-161, 164, 169, 175-186, 197, 198, and 203-216 using the numbering according to SEQ ID NO. 2.

When the agent is used, such a combination of a plurality of proteases results in significantly enhanced cleaning power when it comes to tenacious soling, in particular, soiling that has been burnt on, in particular, milk that has been burned on and/or sugar-containing soiling—such as crème brûlée—that has been burnt on.

A further object contemplated herein is the use of a cleaning agent described herein, preferably a dishwashing agent, especially a machine dishwashing agent in a cleaning method, preferably a dishwashing method, especially in a machine dishwashing method, preferably the use in order to improve the cleaning power, in particular the cleaning power on protease-sensitive soiling, on hard surfaces, in particular dishes when cleaning the same, preferably in an automatic dishwasher, in particular burnt-on, tenacious soiling, inter alia, also at temperatures that are lower than the customarily used temperatures.

A further object contemplated herein is a cleaning method, preferably a dishwashing method, especially a machine dishwashing method, in which a cleaning agent described herein, preferably a dishwashing agent, especially a machine dishwashing agent, is used in particular for the purpose of improving the cleaning power when it comes to burnt-on, protease-sensitive soiling. In various embodiments contemplated herein, temperatures that are lower than the customarily used temperatures are used in the dishwashing method.

“Low temperatures” or “temperatures that are lower than the customarily used temperatures,” as used herein in the context of dishwashing methods, preferably refers to temperatures below about 60° C., in particular below about 55° C., still more preferably about 50° C. or lower, particularly preferably about 45° C. or lower, and most preferably about 40° C. or lower. This temperature information refers to the temperatures used in the washing steps.

These and further aspects, features, and advantages of the various embodiments become apparent to a person skilled in the art when studying the following detailed description and claims. Every feature from one aspect of the various embodiments may be used in any other aspect of the various embodiments. Moreover, it shall be readily understood that the examples contained herein are intended to describe and illustrate the various embodiments, but do not limit the same, and in particular the various embodiments are not limited to these examples. All percentage information is percent by weight, unless indicated otherwise. Numerical ranges indicated in the format “from x to y” include the mentioned values. If several preferred numerical ranges are indicated in this format, it shall be readily understood that all ranges resulting from the combination of the different end points are likewise covered.

The proteases used are alkaline serine proteases. They act as non-specific endopeptidases, which is to say they hydrolyze arbitrary acid amide bonds that lie in the interior of peptides or proteins, thereby causing the decomposition of protein-containing soiling on the goods to be cleaned. The pH optimum thereof is usually in the distinctly alkaline range.

The sequences of the mature protease subtilisin 309 from Bacillus lentus, which is sold under the trade name Savinase® by Novozymes A/S, Bagsvaerd, Denmark, or of the mature protease from Bacillus alkalophilus PB92 (wild type) are indicated in SEQ ID NO. 1 and SEQ ID NO. 2, respectively.

“Different,” as used herein with reference to the proteases, refers to proteases that differ in terms of the amino acid sequence thereof. In various embodiments, proteases that are different from each other originate from different types of organisms or differ from each other by mutations, for example those created artificially.

“Variant,” as used herein in the context of proteases, refers to natural or artificially created variations of a native protease which have an amino acid sequence that is modified compared to the reference form. Such a variant may have single or multiple point mutations, which is to say substitutions of one amino acid naturally occurring at the corresponding position by another, insertions (introduction of one or more amino acids), and/or deletions (removal of one or more amino acids), in particular one or more point mutations. Such variants preferably have at least about 50%, preferably about 60% or more, still more preferably about 70%, about 80%, about 90%, about 100%, or more of the enzyme activity of the reference form. In various embodiments, such a variant has an amino acid sequence that is at least about 70%, preferably about 75%, about 80%, about 85%, about 90%, about 95%, about 96%, about 97%, about 98%, or about 99% identical to the sequence serving as the reference across the entire length thereof. The variants preferably have the same length as the reference sequence. Variants may stand out compared to the reference form by having improved properties, such as higher enzyme activity, higher stability, altered substrate specificity, and the like. Only variants exhibiting protease activity are used.

The identity of nucleic acid or amino acid sequences is determined by sequence comparison. This sequence comparison is based on the BLAST algorithm, which is established and commonly used in the prior art (see, for example, Altschul, S. F., Gish, W., Miller, W., Myers, E. W. & Lipman, D J. (1990) “Basic local alignment search tool.” J. Mol. Biol. 215:403-410, and Altschul, Stephan F., Thomas L. Madden, Alejandro A. Schaffer, Jinghui Zhang, Hheng Zhang, Webb Miller, and David J. Lipman (1997): “Gapped BLAST and PSI-BLAST: a new generation of protein database search programs”; Nucleic Acids Res., 25, p. 3389-3402), and is done principally by associating together similar sequences of nucleotides or amino acids in the nucleic acid or amino acid sequences. Tabular mapping of the relevant positions is referred to as alignment. Another algorithm available in the prior art is the FASTA algorithm.

Such a comparison also allows for information to be provided about the similarity of the compared sequences to one another. The similarity is usually indicated in percent identity, i.e., the proportion of identical nucleotides or amino acid residues therein, or of positions corresponding to one another in an alignment. The broader concept of homology refers, in the case of amino acid sequences, to conserved amino acid substitutions, i.e., amino acids that have similar chemical activity because said amino acids exert mostly similar chemical activities within the protein. Hence, the similarity of the compared sequences can also be indicated by percent homology or percent similarity. Identity and/or homology can be indicated over entire polypeptides or genes, or only over individual regions. Homologous or identical regions of different nucleic acid or amino acid sequences are thus defined by matches in the sequences. Such regions often have identical functions. Such regions may be small and include only a small number of nucleotides or amino acids. Often, such small regions perform functions that are essential for the overall activity of the protein. It may therefore be practical to indicate sequence matches only over individual—optionally, small—regions. Unless otherwise specified, however, identities or homologies given in the present patent application refer to the total length of the respectively indicated nucleic acid or amino acid sequence.

The protease used as the first protease as contemplated herein is one that comprises an amino acid sequence which is at least about 80% identical to the amino acid sequence indicated in SEQ ID NO. 1 over the entire length thereof, and comprises at least one amino acid substitution at one of the positions 9, 15, 66, 212, and 239 using the numbering according to SEQ ID NO. 1.

The first protease used may thus be a variant of subtilisin 309 from Bacillus lentus including the amino acid sequence indicated in SEQ ID NO. 1, which is at least about 80% identical to the amino acid sequence indicated in SEQ ID NO. 1 over the entire length thereof and includes at least one amino acid substitution at one of the positions 9, 15, 66, 212, and 239 using the numbering according to SEQ ID NO. 1. Preferably proteases are used which have an amino acid substitution at two, preferably three, in particular four, most particularly preferably five of the above-mentioned positions.

Other variants that may be used are those which include an amino acid sequence that is at least about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% identical to the amino acid sequence indicated in SEQ ID NO. 3, wherein positions 9, 15, 66, 212, and 239 are invariable, which is to say the amino acid at these positions corresponds to the corresponding amino acid in SEQ ID NO. 3.

It is particularly preferable to use a variant including at least one, preferably two, in particular three, particularly preferably four, or most particularly preferably five of the amino acid substitutions selected from S9R, A15T, V66A, N212D, and Q239R, based on the numbering according to SEQ ID NO. 1. The following combinations are preferred: S9R+V66A+N212D+Q239R, S9R+A15T+N212D+Q239R, S9R+A15T+V66A+Q239R, S9R+A15T+V66A+N212D, A15T+V66A+N212D+Q239R; S9R+A15T+V66A, S9R+A15T+N212D, S9R+A15T+Q239R, S9R+N212D+Q239R, S9R+V66A+N212D, S9R+V66A+Q239R, A15T+V66A+N212D, A15T+V66A+Q239R, A15T+N212D+Q239R, V66A+N212D+Q239R; S9R+A15T, S9R+V66A, S9R+N212D, S9R+Q239R, A15T+V66A, A15T+N212D, A15T+Q239R, V66A+N212D, V66A+Q239R, N212D+Q239R. A variant that comprises all of the above changes has the amino acid sequence indicated in SEQ ID NO. 3 (S9R+A15T+V66A+N212D+Q239R).

The second protease is different from the first protease, which is to say a protease that is covered both by the definition of the first protease and that of the second protease cannot simultaneously be considered a first protease and a second protease.

As contemplated herein, the second protease comprises an amino acid sequence which is at least 80% identical to the amino acid sequence specified in SEQ ID NO. 2 over the entire length and has at least one amino acid substitution at one of the following positions 32, 33, 48-54, 58-62, 94-107, 116, 123-133, 150, 152-156, 158-161, 164, 169, 175-186, 197, 198, and 203-216 using the numbering according to SEQ ID NO 2.

Further variants that may be used are those that have an amino acid sequence that is at least about 80% and increasingly preferably at least about 81%, about 82%, about 83%, about 84%, about 85%, about 86%, about 87%, about 88%, about 89%, about 90%, about 90.5%, about 91%, about 91.5%, about 92%, about 92.5%, about 93%, about 93.5%, about 94%, about 94.5%, about 95%, about 95.5%, about 96%, about 96.5%, about 97%, about 97.5%, about 98%, about 98.5%, and about 99% identical to the amino acid sequence indicated in SEQ ID NO. 2, and bears an amino acid substitution at at least one of the following positions 32, 33, 48-54, 58-62, 94-107, 116, 123-133, 150, 152-156, 158-161, 164, 169, 175-186, 197, 198, and 203-216, using the numbering according to SEQ ID NO. 2.

A preferred second protease that can be used is a variant of the protease which has the amino acid sequence indicated in SEQ ID NO. 2 and is at least 80% identical to the amino acid sequence indicated in SEQ ID NO. 2 over the entire length thereof and includes at least one amino acid substitution at one of the positions 116, 126, 127, 128, and 160, using the numbering according to SEQ ID NO. 2. It is preferable to use proteases that have an amino acid substitution at two, preferable three or more, in particular, four of the aforementioned positions.

Further variants that may be used are those that have an amino acid sequence that is at least up to about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% identical to the amino acid sequence indicated in SEQ ID NO. 2, and bears at least one amino acid substitution at at least one of the following positions 116, 126, 127, 128, and 160.

Particularly preferably, the second protease has an amino acid sequence that is at least 80% identical to the amino acid sequence indicated in SEQ ID NO. 2 over the total length thereof and that has one of the following amino acid substitutions using the numbering according to SEQ ID NO. 2:

(i) G116V+S126L+P127Q+S128A

(ii) G116V+S126N+P127S+S128A+S160D

(iii) G116V+S126L+P127Q+S128A+S160D

(iv) G116V+S126V+P127E+S128K

(v) G116V+S126V+P127M+A160D

(vi) S128T

(vii) G116V+S126F+P127L+S128T

(viii) G116V+S126L+P127N+S128V

(ix) G116V+S126F+P127Q

(x) G116V+S126V+P127E+S128K+S160D

(xi) G116V+S126R+P127S+S128P

(xii) S126R+P127Q+S128D

(xiii) S126C+P127R+S128D; or

(xiv) S126C+P127R+S128G

The second protease preferably comprises an amino acid sequence that has at least one, preferably more, in particular, all of the following amino acid substitutions G116V, S126L, P127Q, and/or S128A using the numbering according to SEQ ID NO. 2 and is at least about 80%, about 81%, about 82%, about 83%, about 84%, about 85%, about 86%, about 87%, about 88%, about 89%, about 90%, about 90.5%, about 91%, about 91.5%, about 92%, about 92.5%, about 93%, about 93.5%, about 94%, about 94.5%, about 95%, about 95.5%, about 96%, about 96.5%, about 97%, about 97.5%, about 98%, about 98.5%, and about 99%, in particular, up to about 100% identical at all other positions to the amino acid sequence indicated in SEQ ID NO. 2 over the entire length thereof. Particularly preferred is a protease that has an amino acid sequence that can be obtained from the amino acid sequence having the SEQ ID NO. 2 by the amino acid substitutions G116V, S126L, P127Q, and S128A, using the numbering according to SEQ ID NO. 2.

Preferred combinations of proteases are, in particular, the combination of the protease having the amino acid sequence indicated in SEQ ID NO. 3 with a protease that has an amino acid sequence that has at least one, preferably more, in particular, all of the following amino acid substitutions G116V, S126L, P127Q, and/or S128A, using the numbering according to SEQ ID NO. 2, and otherwise has the amino acid sequence indicated in SEQ ID NO. 2.

In different embodiments, these combinations of proteases are used in a mass ratio of about 10:1 to about 1:10, preferably about 5:1 to about 1:5, in particular, about 2:1 to about 1:2, e.g., about 2:3 to about 3:2, particularly preferably equal parts, based on active protein.

It is particularly preferable to use the protease that has the amino acid sequence indicated in SEQ ID NO. 3 with the above-described second protease, in particular, which has an amino acid sequence that has at least one, preferably more, in particular, all of the following amino acid substitutions G116V, S126L, P127Q, and/or S128A, using the numbering according to SEQ ID NO. 2, and is at all other positions up to about 100% identical to the amino acid sequence indicated in SEQ ID NO. 2 over the entire length thereof, in a mass ratio of about 10:1 to about 1:10, preferably about 5:1 to about 1:5, in particular, about 2:1 to about 1:2, e.g., about 2:3 to about 3:2, and particularly preferably in equal parts. Surprisingly, the combinations of different proteases described herein have the property of improving the power of the cleaning agent, preferably of the dishwashing agent, by resulting in improved cleaning power when it comes to tenacious, burnt soiling. The increase in cleaning power can also be observed at low temperatures, which is to say temperatures that are lower than those customarily used in dishwashing methods, as defined above. This makes it possible to carry out the cleaning method, preferably the machine dishwashing method, at lower temperatures and nonetheless preserve the good cleaning power.

The improvement in cleaning power shall in general be understood to mean that the removal of soiling, in particular burnt soiling, from hard surfaces, in particular dishes, is noticeably improved by use of the cleaning agents, in particular, dishwashing agents described herein, to clean the same, preferably in an automatic dishwasher, in comparison to the use of cleaning agents, preferably dishwashing agents, that do not contain the enzyme combinations described herein.

The enzymes to be used can furthermore be formulated together with by-products, such as from fermentation, or with stabilizers.

The cleaning agents, in particular dishwashing agents, preferably contain each protease in a quantity from about 1×10⁻⁸ to about 5 wt %, based on the respective active protein. Preferably about 0.001 to about 2 wt %, more preferably about 0.005 to about 1.5 wt %, still more preferably about 0.01 to about 1 wt %, and particularly preferably about 0.01 to about 0.5 wt % of each enzyme is present in these agents.

In particularly preferred embodiments, the first protease is used in the agents described herein in a total quantity from about 0.01 to about 1 wt %, preferably about 0.025 to about 0.5 wt %, based on active protein. Similarly, the second protease is preferably used in a total quantity from about 0.005 to about 0.75 wt %, preferably about 0.01 to about 0.5 wt %, based on active protein. A preferred first protease is about 0.025 to about 0.5 wt % of the protease having the sequence of SEQ ID NO. 3, and a preferred second protease is about 0.01 to about 0.5 wt % of the protease that has an amino acid sequence that has at least one, preferably more, in particular, all of the following amino acid substitutions G116V, S126L, P127Q, and/or S128A, using the numbering according to SEQ ID NO. 2, and is up to about 100% identical at all other positions to the amino acid sequence indicated in SEQ ID NO. 2 over the entire length thereof. In liquid formulations, the enzymes are preferably used in the form of liquid enzyme formulation(s).

The protein concentration can be determined using known methods, such as the BCA method (bicinchoninic acid; 2,2-bichinolyl-4,4′-dicarbonic acid) or the biuret method. The active protein concentration is determined in this regard by a titration of the active centers using a suitable irreversible inhibitor (for proteases, for example, phenylmethylsulfonyl fluoride (PMSF)) and determination of the residual activity (see M. Bender et al., J. Am. Chem. Soc. 88, 24 (1966), pp. 5890-5913).

In particular during storage, the proteases may be protected against damage, such as inactivation, denaturing or disintegration, for example due to physical influences, oxidation, or proteolytic cleavage. Inhibiting proteolysis is particularly preferred in the case of microbial production. The described agents may contain stabilizers for this purpose.

Proteases with cleaning action are generally not provided in the form of the pure protein, but rather in the form of stabilized, storable, and transportable preparations. These preformulated preparations include, for example, solid preparations obtained by way of granulation, extrusion or lyophilization or, in particular in the case of liquid or gel-like agents, solutions of the enzymes, advantageously concentrated to the extent possible, low-hydrate and/or mixed with stabilizers or other auxiliary agents.

Alternatively, the enzymes can be encapsulated, both for the solid and the liquid packaging format, for example by spray drying or extruding the enzyme solution together with a preferably natural polymer, or in the form of capsules, for example those in which the enzymes are enclosed as in a solidified gel, or in those of the core-shell type, in which an enzyme-containing core is coated with a protective layer impervious to water, air, and/or chemicals. Additional active agents, such as stabilizers, emulsifiers, pigments, bleaching agents or dyes can additionally be applied in superimposed layers. Such capsules are applied using methods that are known per se, for example agitation or roll granulation or in fluid bed processes. Such granules are advantageously low-dust, for example by applying polymeric film formers, and storage-stable due to the coating.

It is furthermore possible to formulate two or more enzymes together, so that individual granules have multiple enzyme activities.

As is apparent from the comments above, the enzyme protein forms only a fraction of the total weight of customary enzyme preparations. Preferably used protease preparations contain between about 0.1 and about 40 wt %, preferably between about 0.2 and about 30 wt %, particularly preferably between about 0.4 and about 20 wt %, and in particular between about 0.8 and about 15 wt % of the enzyme protein. The described agents thus preferably comprise such enzyme preparations in each case in quantities from about 0.1 to about 10 wt %, preferably about 0.2 to about 5 wt %, based on the total agent.

The cleaning agents described herein, in particular the preferred machine dishwashing agents, can be of a solid or liquid nature, and in particular be present as powdery solids, in post-compacted particle form, as homogeneous solutions, or suspensions. In a further preferred embodiment contemplated herein, the machine dishwashing agent is present in pre-proportioned form. In a further preferred embodiment contemplated herein, the machine dishwashing agent comprises a plurality of compositions that are spatially separated from each other, whereby it is possible to separate incompatible ingredients from each other, or to offer compositions in combinations, which are used at different points in time in the dishwasher. This is particularly advantageous when the machine dishwashing agents are present in pre-proportioned form. At least one of the compositions may be present in solid form and/or at least one of the compositions may be present in liquid form, wherein the proteases are present in at least one of the compositions, but may also be present in a plurality of compositions.

The agents described herein preferably comprise at least one further component, in particular at least two further components, selected from the group consisting of builders, surfactants, polymers, bleaching agents, bleach catalysts, bleach activators, non-protease enzymes, corrosion inhibitors and glass corrosion inhibitors, disintegration excipients, odorants, and perfume carriers.

Possible ingredients are described hereafter, which can advantageously be used in the cleaning agents described herein, in particular dishwashing agents, preferably machine dishwashing agents.

Advantageously, builders may be used. The builders include in particular zeolites, silicates, carbonates, organic cobuilders and—where ecological bias against their use is absent—also phosphates.

Crystalline layered silicates may be used in the agents described herein. Such cleaning agents, preferably dishwashing agents, in particular machine dishwashing agents, preferably contain a weight fraction of crystalline layered silicate from about 0.1 to about 20 wt %, preferably from about 0.2 to about 15 wt %, and in particular from about 0.4 to about 10 wt %, in each case based on the total weight of these agents.

It is also possible to use the generally known phosphates as builder substances, provided that such use should not be avoided for ecological reasons. Among the plurality of commercially available phosphates, alkali metal phosphates have the greatest significance in the laundry or cleaning agent industry, pentasodium and pentapotassium triphosphate (sodium or potassium tripolyphosphate) being particularly preferred.

Alkali metal phosphates is the term that covers all the alkali metal (in particular, sodium and potassium) salts of the different phosphoric acids, in which a distinction can be made between metaphosphoric acids (HPO₃)ₙ and orthophosphoric acid H₃PO₄, in addition to higher molecular weight representatives. The phosphates combine several advantages: they act as alkali carriers, prevent limescale deposits on machine parts or lime scaling on woven fabrics, and additionally contribute to the cleaning power.

Technically particularly important phosphates are pentasodium triphosphate, Na₅P₃O₁₀ (sodium tripolyphosphate), and the corresponding potassium salt pentapotassium triphosphate, K₅P₃O₁₀ (potassium tripolyphosphate). The sodium potassium tripolyphosphates are preferably used.

If, within the scope of the present application, phosphates are used as substances with washing or cleaning action in the cleaning agents, preferably dishwashing agents, in particular in the machine dishwashing agent, then preferred agents comprise this (these) phosphate(s), preferably alkali metal phosphate(s), particularly preferably pentasodium or pentapotassium triphosphate (sodium or potassium tripolyphosphate), in quantities from about 5 to about 80 wt %, preferably from about 15 to about 75 wt %, and in particular from about 20 to about 70 wt %, in each case based on the weight of the cleaning agent, preferably dishwashing agent, in particular machine dishwashing agent.

Other builders are the alkali carriers. Valid examples of alkali carriers include alkali metal hydroxides, alkali metal carbonates, alkali metal hydrogen carbonates, alkali metal sesquicarbonates, the described alkali silicates, alkali metal silicates and mixtures of the above-mentioned substances, wherein within the meaning contemplated herein preferably the alkali carbonates, in particular sodium carbonate, sodium hydrogen carbonate or sodium sesquicarbonate may be used. A builder system containing a mixture of tripolyphosphate and sodium carbonate is particularly preferred. A builder system containing a mixture of tripolyphosphate and sodium carbonate and sodium silicate is likewise particularly preferred. Due to the low chemical compatibility of the optional alkali metal hydroxides with the remaining ingredients of cleaning agents, in particular dishwashing agents, preferably machine dishwashing agents, compared to other builder substances, they are preferably used only in small quantities or not at all.

The use of carbonate(s) and/or hydrogen carbonate(s), preferably alkali carbonate(s), particularly preferably sodium carbonate, in quantities from about 2 to about 50 wt %, preferably from about 5 to about 40 wt %, and in particular from about 7.5 to about 30 wt %, in each case based on the weight of the agent, preferably machine dishwashing agent, is particularly preferred. Agents that, based on the weight of the machine dishwashing agent, contain less than about 20 wt %, especially less than about 17 wt %, preferably less than about 13 wt %, and in particular less than about 9 wt % carbonate(s) and/or hydrogen carbonate(s), preferably alkali carbonate(s), particularly preferably sodium carbonate, are particularly preferred.

In particular, polycarboxylates/polycarboxylic acids, polymeric polycarboxylates, aspartic acid, polyacetals, dextrins, further organic cobuilders, and phosphonates should be mentioned as organic cobuilders. These substance classes are described hereafter.

Usable organic builder substances are, for example, the polycarboxylic acids that can be used in the form of the free acid and/or of the sodium salts thereof, wherein polycarboxylic acids shall be understood to mean those carboxylic acids that carry more than one acid function. These include, for example, citric acid, adipic acid, succinic acid, glutaric acid, malic acid, tartaric acid, maleic acid, fumaric acid, saccharic acids, nitrilotriacetic acid (NTA), provided that such use is not objectionable for ecological reasons, and mixtures thereof. In addition to the builder effect, the free acids typically also have the property of being an acidifying component and are thus also used as agents to set a lower and milder pH value. In particular, citric acid, succinic acid, glutaric acid, adipic acid, gluconic acid and arbitrary mixtures of these should be mentioned here.

The use of citric acid and/or citrates in these agents has proven to be particularly advantageous for the cleaning and rinsing power of agents described herein. Preferred are therefore cleaning agents, preferably dishwashing agents, particularly preferably machine dishwashing agents, characterized in that the agent contains citric acid or a salt of citric acid, and the weight fraction of the citric acid or of the salt of citric acid especially is more than about 10 wt %, preferably more than about 15 wt %, and in particular between about 20 and about 40 wt %.

Aminocarboxylic acids and/or the salts thereof are another significant class of phosphate-free builders. Particularly preferred representatives of this class are methylglycine diacetic acid (MGDA) or the salts thereof, and glutamine diacetic acid (GLDA) or the salts thereof, or ethylenediamine diacetic acid (EDDS) or the salts thereof. The content of these amino carboxylic acids or of the salts thereof can amount to, for example, between about 0.1 and about 15 wt %, preferably between about 0.5 and about 10 wt %, and in particular between about 0.5 and about 6 wt %. Aminocarboxylic acids and the salts thereof can be used together with the above-mentioned builders, in particular also with the phosphate-free builders.

Suitable builders moreover include polymeric polycarboxylates; for example, these are the alkali metal salts of polyacrylic acid or of polymethacrylic acid, for example those having a relative molar mass from about 500 to about 70,000 g/mol. Suitable polymers are in particular polyacrylates, which preferably have a molar mass from about 2000 to about 20,000 g/mol. Due to the superior solubility thereof, short-chain polyacrylates having molar masses from about 2000 to about 10,000 g/mol, and particularly preferably from about 3000 to about 5000 g/mol, may in turn be preferred from this group.

Also suitable are copolymeric polycarboxylates, in particular those of acrylic acid with methacrylic acid, and of acrylic acid or methacrylic acid with maleic acid.

The (co)polymeric polycarboxylates can be used either as a powder or as an aqueous solution. The content of (co)polymeric polycarboxylates in the cleaning agents, preferably dishwashing agents, in particular machine dishwashing agents, is preferably about 0.5 to about 20 wt %, and in particular about 3 to about 10 wt %.

To improve water solubility, the polymers can also contain allyl sulfonic acids, such as allyloxybenzene sulfonic acid and methallyl sulfonic acid, as a monomer. Further preferred copolymers are those that contain acrolein and acrylic acid/acrylic acid salts or acrolein and vinylacetate as monomers.

Moreover, all compounds that are able to form complexes with alkaline earth ions can be used as builders.

The agents described herein may comprise surfactants, wherein nonionic, anionic, cationic, and amphoteric surfactants are included in the group of surfactants.

All nonionic surfactants known to a person skilled in the art may be used as nonionic surfactants. Suitable nonionic surfactants are, for example, alkyl glycosides of the general formula RO(G)ₓ, where R corresponds to a primary straight-chain or methyl-branched, in particular methyl-branched at the 2-position, aliphatic group having 8 to 22, preferably 12 to 18 carbon atoms, and G is the symbol that denotes a glycose unit having 5 or 6 carbon atoms, preferably glucose. The degree of oligomerization x, which indicates the distribution of monoglycosides and oligoglycosides, is any number between 1 and 10; x is preferably 1.2 to 1.4.

Another class of nonionic surfactants that can preferably be used, which can be used either as the sole nonionic surfactant or in combination with other nonionic surfactants, is alkoxylated, preferably ethoxylated or ethoxylated and propoxylated fatty acid alkyl esters, preferably having 1 to 4 carbon atoms in the alkyl chain.

Nonionic surfactants of the amine oxide type, for example N-cocoalkyl-N,N-dimethylamine oxide and N-tallowalkyl-N,N-dihydroxyethylamine oxide, and of the fatty acid alkanolamide type may also be suitable. The quantity of these nonionic surfactants is preferably no more than that of the ethoxylated fatty alcohols, in particular no more than half thereof.

Further suitable surfactants are polyhydroxy fatty acid amides, also known as PHFA.

Low-foaming nonionic surfactants can be used as preferred surfactants. With particular preference, the cleaning agents, preferably dishwashing agents, in particular machine dishwashing agents contain nonionic surfactants from the group of alkoxylated alcohols. Alkoxylated, advantageously ethoxylated, in particular primary alcohols having preferably 8 to 18 carbon atoms and on average 1 to 12 mol ethylene oxide (EO) per mol of alcohol, in which the alcohol residue can be linear or preferably methyl-branched at the 2-position, or can contain linear and methyl-branched residues in the mixture, such as those usually present in oxo alcohol groups, are preferably used as nonionic surfactants. However, alcohol ethoxylates having linear groups of alcohols of native origin having 12 to 18 carbon atoms, for example of coconut, palm, tallow fatty or oleyl alcohol, and an average of 2 to 8 mol EO per mol of alcohol are particularly preferred. The preferred ethoxylated alcohols include, for example, C₁₂₋₁₄ alcohols having 3 EO or 4 EO, C₉₋₁₁ alcohol having 7 EO, C₁₃₋₁₅ alcohols having 3 EO, 5 EO, 7 EO, or 8 EO, C₁₂₋₁₈ alcohols having 3 EO, 5 EO, or 7 EO, and mixtures thereof, such as mixtures of C₁₂₋₁₄ alcohol having 3 EO and C₁₂₋₁₈ alcohol having 5 EO. The degrees of ethoxylation indicated represent statistical averages that can correspond to an integer or a fractional number for a specific product. Preferred alcohol ethoxylates exhibit a restricted distribution of homologs (narrow range ethoxylates, NRE). In addition to these nonionic surfactants, fatty alcohols having more than 12 EO can also be used. Examples of these are tallow fatty alcohol having 14 EO, 25 EO, 30 EO, or 40 EO.

Nonionic surfactants that have a melting point above room temperature are particularly preferred. Nonionic surfactant(s) having a melting point above about 20° C., preferably above about 25° C., particularly preferably between about 25 and about 60° C., and in particular between about 26.6 and about 43.3° C., is/are particularly preferred.

Surfactants that are preferably to be used come from the groups of alkoxylated nonionic surfactants, in particular ethoxylated primary alcohols.

Anionic surfactants can likewise be used as a component of cleaning agents, preferably dishwashing agents, in particular machine dishwashing agents. These include in particular alkylbenzene sulfonates, (fatty) alkyl sulfates, (fatty) alkyl ether sulfates, and alkanesulfonates. The anionic surfactant content of the agents is usually 0 to about 10 wt %.

Cationic and/or amphoteric surfactants can also be used instead of or in conjunction with the mentioned surfactants. In cleaning agents, in particular dishwashing agents, preferably machine dishwashing agents, the content of cationic and/or amphoteric surfactants is especially less than about 6 wt %, preferably less than about 4 wt %, most particularly preferably less than about 2 wt %, and in particular less than about 1 wt %. Agents that contain no cationic or amphoteric surfactants are particularly preferred.

The group of polymers includes in particular polymers with washing or cleaning action, for example rinse polymers and/or polymers acting as softeners. In addition to nonionic polymers, in general cationic, anionic, and amphoteric polymers can also be used in machine dishwashing agents.

“Cationic polymers” within the meaning contemplated herein are polymers that carry a positive charge in the polymer molecule. This charge can be implemented, for example, by way of (alkyl)ammonium groupings or other positively charged groups that are present in the polymer chain. Particularly preferred cationic polymers come from the groups of quaternized cellulose derivatives, polysiloxanes having quaternary groups, cationic guar derivatives, polymeric dimethyldiallylammonium salts and the copolymers thereof with esters and amides of acrylic acid and methacrylic acid, copolymers of vinylpyrrolidone with quaternized derivatives of dialkylaminoacrylate and -methacrylate, vinylpyrrolidone/methoimidazolinium chloride copolymers, quaternized polyvinyl alcohols, or the polymers described by the INCI names polyquaternium 2, polyquaternium 17, polyquaternium 18, and polyquaternium 27.

“Amphoteric polymers” within the meaning contemplated herein further comprise negatively charged groups or monomer units, in addition to a positively charged group, in the polymer chain. These groups can be, for example, carboxylic acids, sulfonic acids, or phosphonic acids.

Amphoteric polymers that are preferably used come from the group of alkylacrylamide/acrylic acid copolymers, alkylacrylamide/methacrylic acid copolymers, alkylacrylamide/methylmethacrylic acid copolymers, alkylacrylamide/acrylic acid/alkyl aminoalkyl(meth)acrylic acid copolymers, alkylacrylamide/methacrylic acid/alkylaminoalkyl(meth)acrylic acid copolymers, alkylacrylamide/methylmethacrylic acid/alkylaminoalkyl(meth)acrylic acid copolymers, alkylacrylamide/alkyl methacrylate/alkylamino ethyl methacrylate/alkyl methacrylate copolymers, and copolymers of unsaturated carboxylic acids, cationically derivatized unsaturated carboxylic acids, and optionally further ionic or non-ionogenic monomers.

Preferred zwitterionic polymers come from the group of acrylamidoalkyltrialkylammonium chloride/acrylic acid copolymers and the alkali and ammonium salts thereof, acrylamidoalkyltrialkylammonium chloride/methacrylic acid copolymers and the alkali and ammonium salts thereof, and methacroylethylbetaine/methacrylate copolymers.

Cleaning agents, in particular dishwashing agents, preferably machine dishwashing agents, preferably contain the above-mentioned cationic and/or amphoteric polymers in quantities between about 0.01 and about 10 wt %, in each case based on the total weight of the machine dishwashing agent. However, machine dishwashing agents in which the weight fraction of cationic and/or amphoteric polymers is between about 0.01 and about 8 wt %, especially between about 0.01 and about 6 wt %, preferably between about 0.01 and about 4 wt %, particularly preferably between about 0.01 and about 2 wt %, and in particular between about 0.01 and about 1 wt %, based in each case on the total weight of the machine dishwashing agent, are preferred within the scope of the present application.

Bleaching agents can furthermore be used in the cleaning agents, preferably dishwashing agents, in particular machine dishwashing agents. Among the compounds that serve as bleaching agents and yield H₂O₂ in water, sodium percarbonate, sodium perborate tetrahydrate, and sodium perborate monohydrate are of particular importance. Further usable bleaching agents are, for example, peroxypyrophosphates, citrate perhydrates, and peracid salts or peracids that yield H₂O₂, such as perbenzoates, peroxophthalates, diperazelaic acid, phthaloimino peracid, or diperdodecanedioic acid. All further inorganic or organic peroxy bleaching agents known from the prior art to a person skilled in the art may also be used.

Substances that release chlorine or bromine can also be used as bleaching agents. Among the suitable materials releasing chlorine or bromine, for example, heterocyclic N-bromamides and N-chloramides, such as trichloroisocyanuric acid, tribromoisocyanuric acid, dibromoisocyanuric acid, and/or dichloroisocyanuric acid (DICA), and/or the salts thereof with cations such as potassium and sodium can be considered. Hydantoin compounds, such as 1,3-dichloro-5,5-dimethylhydantoin, are likewise suitable.

Cleaning agents, preferably dishwashing agents, in particular machine dishwashing agents that contain about 1 to about 35 wt %, preferably about 2.5 to about 30 wt %, particularly preferably about 3.5 to about 20 wt %, and in particular about 5 to about 15 wt % bleaching agent, preferably sodium percarbonate, are preferred.

The cleaning agents, preferably dishwashing agents, in particular machine dishwashing agents can furthermore contain bleach catalysts. The usable bleach catalysts include, but are not limited to, the group of the bleach-boosting transition metal salts and transition metal complexes, preferably the Mn, Fe, Co, Ru or Mo complexes, particularly preferably from the group of the manganese and/or cobalt salts and/or complexes, in particular the cobalt (ammine) complexes, the cobalt (acetate) complexes, the cobalt (carbonyl) complexes, the chlorides of cobalt or manganese, manganese sulfate and the complexes of manganese with 1,4,7-trimethyl-1,4,7-triazacyclononane (Mn₃-TACN) or 1,2,4,7-tetramethyl-1,4,7-triazacyclononane (Mna-TACN).

Cleaning agents, preferably dishwashing agents, in particular machine dishwashing agents that contain about 0.001 to about 1 wt %, preferably about 0.01 to about 0.1 wt % bleach catalyst, preferably an Mn complex, in particular a complex of manganese with 1,4,7-trimethyl-1,4,7-triazacyclononane (Mn₃-TACN) or 1,2,4,7-tetramethyl-1,4,7-triazacyclononane (Mn₄-TACN) are preferred.

In various embodiments contemplated herein, the cleaning agents, preferably dishwashing agents, in particular machine dishwashing agents, additionally contain at least one bleach activator. Compounds that, under perhydrolysis conditions, yield aliphatic peroxocarboxylic acids having preferably 1 to 10 carbon atoms, in particular 2 to 4 carbon atoms, and/or optionally substituted perbenzoic acid, can be used as bleach activators. Out of all bleach activators known to a person skilled in the art from the prior art, polyacylated alkylenediamines, in particular tetra acetyl ethylene diamine (TAED), acylated triazine derivatives, in particular 1,5-diacetyl-2,4-dioxohexahydro-1,3,5-triazine (DADHT), acylated glycolurils, in particular tetraacetyl glycoluril (TAGU), N-acylimides, in particular N-nonanoyl succinimide (NOSI), acylated phenolsulfonates, in particular n-nonanoyl or iso-nonanoyl oxybenzene sulfonate (n- or iso-NOBS), are particularly preferred. It is also possible to use combinations of conventional bleach activators. These bleach activators are preferably used in quantities of up to about 10 wt %, in particular about 0.1 wt % to about 8 wt %, particularly about 2 to about 8 wt %, and particularly preferably about 2 to about 6 wt %, based in each case on the total weight of the bleach activator-containing agent.

The agents contemplated herein preferably contain at least one additional enzyme preparation or enzyme composition, which contains one or more non-protease enzymes. Such enzymes include, without being limited thereto, amylases, lipases, cellulases, hemicellulases, mannanases, pectin-cleaving enzymes, tannanases, xylanases, xanthanases, β-glycosidases, carrageenanases, perhydrolases, oxidases, oxidoreductases, and the mixtures thereof. Preferred enzymes comprise in particular amylases, in particular α-amylases, cellulases, lipases, hemicellulases, in particular pectinases, mannanases, β-glucanases, and the mixtures thereof. Amylases and/or lipases and the mixtures thereof are particularly preferred. These enzymes are, in principle, of natural origin; proceeding from the natural molecules, improved variants are available for use in washing or cleaning agents and can be used in a correspondingly preferred fashion.

The information provided on quantities and formulation forms in connection with the proteases used apply mutatis mutandis also to all further above-described enzymes.

Glass corrosion inhibitors prevent the appearance of clouding, streaking, and scratching, but also iridescence of the glass surface of machine-cleaned glassware. Preferred glass corrosion inhibitors come from the group of magnesium and zinc salts and of the magnesium and zinc complexes. Within the scope contemplated herein, the content of zinc salt in cleaning agents, preferably dishwashing agents, in particular machine dishwashing agents, is especially between about 0.1 and about 5 wt %, preferably between about 0.2 and about 4 wt %, and in particular between about 0.4 and about 3 wt %, or the content of zinc in oxidized form (calculated as Zn²⁺) is between about 0.01 and about 1 wt %, especially between about 0.02 and about 0.5 wt %, and in particular between about 0.04 and about 0.2 wt %, in each case based on the total weight of the glass corrosion inhibitor-containing agent.

So as to facilitate the breakdown of prefabricated shaped bodies, it is possible to incorporate disintegration excipients, known as tablet disintegrants, into these agents in order to shorten breakdown times. Tablet disintegrants or breakdown accelerators are understood to mean excipients that ensure rapid breakdown of tablets in water or other media, and the quick release of the active agents. Disintegration excipients can preferably be used in quantities from about 0.5 to about 10 wt %, preferably about 3 to about 7 wt %, and in particular about 4 to about 6 wt %, in each case based on the total weight of the agent comprising the disintegration excipient.

Individual odorous substance compounds, such as synthetic products of the ester, ether, aldehyde, ketone, alcohol, and hydrocarbon types, can be used within the scope contemplated herein as perfume oils or odorants. Preferably, however, mixtures of different odorous substances are used, which together produce an appealing odorous note. Such perfume oils can also contain natural odorous substance mixtures such as those accessible from plant sources, for example pine, citrus, jasmine, patchouli, rose, or ylang ylang oil. Cleaning agents described herein, preferably dishwashing agents, in particular machine dishwashing agents can be formulated in a variety of ways. The agents can be present in solid or liquid presentation forms or as a combination of solid and liquid presentation forms. Suitable solid presentation forms are, in particular, powders, granules, extrudates, compactates, in particular tablets. The liquid presentation forms based on water and/or organic solvents can be present in thickened form, in the form of gels. Agents described herein can be formulated in the form of single-phase or multi-phase products.

Cleaning agents described herein, preferably dishwashing agents, in particular machine dishwashing agents, are preferably preformulated as dosing units. These dosing units preferably comprise the quantity of substances with washing or cleaning action necessary for one cleaning cycle.

The cleaning agents described herein, preferably dishwashing agents, in particular machine dishwashing agents, in particular the prefabricated dosing units, particularly preferably comprise a water-soluble wrapping.

The water-soluble wrapping is preferably formed of a water-soluble film material selected from the group consisting of polymers or polymer mixtures. The wrapping may be formed of one layer or of two or more layers of the water-soluble film material. The water-soluble film material of the first layer and that of the further layers, if such are present, may be the same or different. Films that can be bonded and/or sealed after they have been filled with the agent to form packagings, such as tubes or cushions, are particularly preferred.

It is preferable for the water-soluble wrapping to comprise polyvinyl alcohol or a polyvinyl alcohol copolymer. Water-soluble wrappings comprising polyvinyl alcohol or a polyvinyl alcohol copolymer exhibit good stability and sufficiently high water solubility, in particular cold water solubility.

Suitable water-soluble films for producing the water-soluble wrapping are preferably based on a polyvinyl alcohol, or a polyvinyl alcohol copolymer, having a molecular weight in the range from about 10,000 to about 1,000,000 gmol⁻¹, preferably from about 20,000 to about 500,000 gmol⁻¹, particularly preferably from about 30,000 to about 100,000 gmol⁻¹, and in particular from about 40,000 to about 80,000 gmol⁻¹.

The polyvinyl alcohol is typically produced by the hydrolysis of polyvinyl acetate, since the direct synthesis pathway is not possible. The same applies to polyvinyl alcohol copolymers produced accordingly from polyvinyl acetate copolymers. It is preferred if at least one layer of the water-soluble wrapping comprises a polyvinyl alcohol having a degree of hydrolysis of about 70 to about 100 mol %, preferably about 80 to about 90 mol %, particularly preferably about 81 to about 89 mol %, and in particular about 82 to about 88 mol %.

Additionally, a polymer selected from the group consisting of (meth)acrylic acid-containing (co)polymers, polyacrylamides, oxazoline polymers, polystyrene sulfonates, polyurethanes, polyesters, polyethers, polylactic acid, or mixtures of the above polymers can be added to a polyvinyl alcohol-containing film material that is suitable for producing the water-soluble wrapping. A preferred additional polymer is polylactic acids.

Preferred polyvinyl alcohol copolymers comprise, in addition to vinyl alcohol, dicarboxylic acids as further monomers. Suitable dicarboxylic acids are itaconic acid, malonic acid, succinic acid, and mixtures thereof, itaconic acid being preferred.

Likewise, preferred polyvinyl alcohol copolymers include an ethylenically unsaturated carboxylic acid, the salt thereof, or the ester thereof, in addition to vinyl alcohol. In addition to vinyl alcohol, such polyvinyl alcohol copolymers particularly preferably comprise acrylic acid, methacrylic acid, acrylic acid esters, methacrylic acid esters, or mixtures thereof.

It may be preferred for the film material to contain further additives. For example, the film material may contain plasticizers such as dipropylene glycol, ethylene glycol, diethylene glycol, propylene gylcol, glycerol, sorbitol, mannitol, or mixtures thereof. Examples of further additives include release aids, fillers, cross-linking agents, surfactants, antioxidants, UV absorbers, anti-blocking agents, non-stick agents, or mixtures thereof.

Suitable water-soluble films for use in the water-soluble wrappings of the water-soluble packagings contemplated herein are films sold by MonoSol LLC, for example, by the designation M8630, C8400, or M8900. Other suitable films include films by the designation Solublon® PT, Solublon® GA, Solublon® KC or Solublon® KL from Aicello Chemical Europe GmbH, or the VF-HP films from Kuraray.

A further object as contemplated herein is also the corresponding use of the agents described herein. A dishwashing method, in particular a machine dishwashing method, is also contemplated in which an agent as described herein is used. A further object of the present application is therefore moreover a method for cleaning dishes in a dishwasher, in which the agent is dispensed into the interior of a dishwasher while a dishwashing program is being executed, before the main washing cycle begins, or during the course of the main washing cycle. The agent may be manually dispensed or introduced into the interior of the dishwasher, but preferably the agent is dispensed into the interior of the dishwasher by means of the dosing chamber. In various embodiments as contemplated herein, the (washing) temperature in such dishwashing methods is preferably about 50° C. or lower, particularly preferably about 45° C. or lower, still more preferably about 40° C. or lower.

A typical basic formulation for a machine dishwashing agent that can preferably be used, for example, in tablet form, comprises the following materials:

sodium tripolyphosphate about 20 to about 50 wt %
sodium carbonate about 10 to about 30 wt %
sodium percarbonate about 5 to about 18 wt %
bleach activator about 0.5 to about 5 wt %
bleach catalyst about 0.01 to about 1 wt %
sulfopolymer about 2.5 to about 15 wt %
polycarboxylate about 0.1 to about 10 wt %
nonionic surfactant about 0.5 to about 10 wt %
phosphonate about 0.5 to about 5 wt %
proteases about 0.1 to about 5 wt %
amylase about 0.1 to about 5 wt %
wherein the information in wt % in each case is based on the total agent. Instead of tripolyphosphate, or a portion of the tripolyphosphate, it is in particular possible to also use about 10 to about 50 wt % citrate or MGDA or GLDA or EDDS or mixtures of two or three of these substances in the formula.

### Examples

### Example 1: Improving the Protease Performance During Automatic Dishwashing

TABLE 1
Composition of the machine dishwashing agent

| | Base |
|---|---|
| phosphate (wt %) | 35.9 |
| sodium carbonate (wt %) | 12.2 |
| phosphonate (wt %) | 2.4 |
| sulfonic acid group-containing | 7.9 |
| polymer (wt %) | |
| polyacrylate (wt %) | 4.6 |
| nonionic surfactants (wt %) | 6.1 |
| percarbonate (wt %) | 14.6 |
| TAED (wt %) | 2.3 |
| bleach catalyst (wt %) | 1.0 |
| polycarboxylate (wt %) | 1.5 |
| sodium silicate/polycarboxylate (wt %) | 3.9 |
| enzyme composition (amylase) (wt %) | 1.0 |
| zinc acetate (wt %) | 0.2 |
| remainder (perfume, dyes, protease or | up to 100% |
| protease mixture, etc.) (wt %) | |

### Example 1: Cleaning Power on Crème Brûlée

In a Miele G698 dishwasher, china plates containing soiling of black tea, egg yolk, ground meat, and crème brûlée were washed at 50° C. (“Normal” cycle) and 21° dH using a solid dishwashing tablet (20 g; for composition, see table 1) comprising various individual proteases (comparison experiments V1 and V2) or protease combinations (M1). The crème brûlée soiling served as tenacious soiling. For this purpose, the ready-made crème brûlée mixture from Debic was heated in a pot to 60° C., and 3.5 g was applied in each case to a dessert plate using a brush and allowed to dry at room temperature for 2 hours. The plates were then placed into a cold oven (Binder) and heated to 140° C. within 1 hour. The crème brûlée soiling was then burnt-in for 2 hours in the oven at 140° C.

The cleaning power was visually determined according to IKW after each washing cycle (evaluation from 1 to 10; the higher the value, the better the power; differences of at least 1 are significant). The results for the tested formulas are listed in table 2 as arithmetic means. Higher values indicate better cleaning power.

The quantity of the corresponding protease preparation (individual proteases, V1 and V2) or of the mixtures thereof (M1; mass ratio of protease V1:V2 is 2:3) added to the formulation according to table 1 was such that the total protease quantity in the formation was 0.24 wt %.

TABLE 2
Cleaning power on crème brûlée

| Protease/protease | | | | |
|---|---|---|---|---|
| combination | Tea | Egg yolk | Starch | Crème Brûlée |
| M1: | 10.0 | 9.5 | 9.3 | 8.6 |
| V1: | 9.5 | 8.7 | 8.7 | 6.2 |
| V2: | 6.0 | 9.6 | 9.7 | 7.6 |

V1: Base + 1. Protease: (enzyme according to SEQ ID NO. 3)
V2: Base + 2. Protease (protease having an amino acid sequence having the following amino acid substitutions G116V, S126L, P127Q, and S128A, using the numbering according to SEQ ID NO. 2, and otherwise having the amino acid sequence indicated in SEQ ID NO. 2)
M1: Base + mixture of the enzyme according to V1 and the enzyme according to V2, in a ratio of 2:3, based on the protease content in wt %

As is apparent in table 2, the first protease (V1) exhibits very good cleaning power when it comes to tea soiling, while cleaning power is only mediocre when it comes to crème brûlée. In contrast, it is apparent that the second protease (V2) has good performance when it comes to crème brûlée, while performance is only mediocre when it comes to tea.

It is clearly apparent in table 2 that the combination of two different proteases results in a considerable improvement of the cleaning power (M1).

### Example 2: Cleaning Power on Milk

In a Bosch SMS86 dishwasher, china plates containing soiling of milk were washed at 40° C. (“Gentle 40” program) and 21° dH using a solid dishwashing tablet. The quantity of the corresponding protease preparation (individual proteases, V1 and V2) or of the mixtures thereof (M1; mass ratio of protease V1:V2 is 5:1) added to the formulation according to table 1 was such that the total protease quantity in the formation was 0.24 wt %. The milk soiling was produced by burning on milk with a fat content of 1.5% in a microwave oven.

TABLE 3
Cleaning power on milk

| Protease/protease combination | Milk |
|---|---|
| M1 (V1 + V2, ratio 5:1) | 7.3 |
| V1: | 6.6 |
| V2: | 6.7 |

V1: Base + 1. Protease: (enzyme according to SEQ ID NO. 3)
V2: Base + 2. Protease (protease having an amino acid sequence having the following amino acid substitutions G116V, S126L, P127Q, and S128A, using the numbering according to SEQ ID NO. 2, and otherwise having the amino acid sequence indicated in SEQ ID NO. 2)
M1: Base + mixture of the enzyme according to V1 and the enzyme according to V2, in a ratio of 5:1, based on the protease content in wt %

It is clearly apparent in table 3 that the combination as contemplated herein leads to a higher cleaning power on milk.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A cleaning agent comprising at least one first protease and one second protease, wherein:
a. the first protease comprises an amino acid sequence which is at least about 80% identical to the amino acid sequence specified in SEQ ID NO. 1 over the entire length and has at least one amino acid substitution at one of the positions 9, 15, 66, 212, and 239 using the numbering according to SEQ ID NO. 1, and
b. the second protease comprises an amino acid sequence which is at least about 80% identical to the amino acid sequence specified in SEQ ID NO. 2 over the entire length and has at least one amino acid substitution at one of the positions 32, 33, 48-54, 58-62, 94-107, 116, 123-133, 150, 152-156, 158-161, 164, 169, 175-186, 197, 198, and 203-216 using the numbering according to SEQ ID NO. 2.

2. The cleaning agent according to claim 1, wherein the first protease has at least one amino acid substitution selected from the group consisting of S9R, A15T, V66A, N212D, and Q239R, using the numbering according to SEQ ID NO. 1.

3. The cleaning agent according to claim 1, wherein the first protease has an amino acid sequence according to SEQ ID NO. 3.

4. The cleaning agent according to claim 1, wherein the second protease has at least one amino acid substitution at one, two, three, or four of the positions 116, 126, 127, 128, and 160 using the numbering according to SEQ ID NO. 2.

5. The cleaning agent according to claim 4, wherein the second protease has at least one of the amino acid substitution G116V, S126L, P127Q, and/or S128A using the numbering according to SEQ ID NO. 2.

6. The cleaning agent according to claim 1, wherein the first protease and the second protease are present in a mass ratio of about 10:1 to about 1:10.

7. The cleaning agent according to claim 1, wherein the weight fraction of each of the proteases, based on the corresponding active protein, is from about 1×10⁻⁸ to about 5 wt %.

8. The cleaning agent according to claim 1, further comprising at least one further component selected from the group of builders, surfactants, polymers, bleaching agents, bleach catalysts, bleach activators, non-protease enzymes, corrosion inhibitors, glass corrosion inhibitors, disintegration excipients, odorants, and perfume carriers.

9. The cleaning agent according to claim 1, wherein the cleaning agent is a dishwashing agent.

10. The cleaning agent according to claim 9, wherein
(1) the dishwashing agent exists in a solid form,
(2) optionally, the dishwashing agent exists in a pre-proportioned form, and
(3) optionally, the dishwashing agent comprises a plurality of compositions that are spatially separated from one another.

11. A method for cleaning dishes in an automatic dishwasher, wherein the method comprises introducing the cleaning agent according to claim 1 into the automatic dishwasher.

12. The method for cleaning dishes according to claim 11, wherein the cleaning agent is dispensed into the interior of the dishwasher while a dishwashing program is being executed, before a main washing cycle begins, or during the course of the main washing cycle.

13. (canceled)

14. The cleaning agent according to claim 5, wherein the second protease comprises an amino acid sequence which is at least 90% identical to the amino acid sequence specified in SEQ ID NO. 2 over the entire length.

15. The cleaning agent according to claim 14, wherein the second protease comprises an amino acid sequence which is up to 100% identical to the amino acid sequence specified in SEQ ID NO. 2 over the entire length.

16. The cleaning agent according to claim 5, wherein the second protease has all of the amino acid substitutions G116V, S126L, P127Q, and/or S128A using the numbering according to SEQ ID NO. 2.

17. The cleaning agent according to claim 7, wherein the weight fraction of each of the proteases, based on the corresponding active protein, is from about 0.01 to about 0.5 wt %.

18. The cleaning agent according to claim 9, wherein the cleaning agent is a machine dishwashing agent.

19. The cleaning agent according to claim 9, wherein the dishwashing agent exists in a pre-proportioned form.

20. The cleaning agent according to claim 9, wherein the dishwashing agent comprises a plurality of compositions that are spatially separated from one another.


SEQUENCE LISTING (3 sequences)

SEQ ID NO: 1 - protein, 269 aa, Bacillus lentus
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 2 - protein, 269 aa, Bacillus alkalophilus
AQSVPWGISRVQAPAAHNRGLTGSGVKVAVLDTGISTHPDLNIRGGASFVPGEPSTQDGNGHGTHVAGTIAALNNSIGVLGVAPNAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 3 - protein, 269 aa, artificial
  variant of subtilisin 309
AQSVPWGIRRVQAPTAHNRGLTGSGVKVAVLDTGISTHPDLNIRGGASFVPGEPSTQDGNGHGTHAAGTIAALNNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLDGTSMATPHVAGAAALVKQKNPSWSNVRIRNHLKNTATSLGSTNLYGSGLVNAEAATR